(19) United States
(12) Patent Application Publication
(10) Pub. No.: US 2004/0137072 A1
(43) Pub. Date: Jul. 15, 2004

(54) Method for increasing calcium absorption and bone mineral density through the supplementation of bovine colostrum

(76) Inventor: Richard Cockrum, Perry, IA (US)

Correspondence Address: Davis, Brown, Koehn, Shors & Roberts, P.C., The Financial Center, Suite 2500, 666 Walnut Street, Des Moines, IA, 50309-3993, US

(21) Appl. No.: 10/340,952

(22) Filed: Jan. 13, 2003

(51) Int. Cl. A61K 35/20

(52) U.S. Cl. 424/535

(57) ABSTRACT

The present invention pertains to a method for increasing calcium absorption and decreasing the bone loss in humans by administering to a human an effective amount of a calcium supplement in conjunction with a colostrum supplement. The supplement administered preferably comprises bovine colostrum as the colostrum supplement.

### BACKGROUND OF THE INVENTION

1. Field of the Invention

The present invention relates generally to a method for increasing calcium absorption in humans and hence bone mineral density through the supplementation of bovine colostrum and, more specifically, to a method for increasing calcium absorption and bone mineral density in humans through the ingestion of a calcium supplement in conjunction with a bovine colostrum supplement.

2. Background

In addition to physical activities, calcium nutrition influences the health of humans and in particular of women during all stages of life (Rourke et al., 1998; and Prentice 2000). Calcium malabsorption and calcium deficiency are frequently considered important contributing factors for bone diseases (Norris et al., 2001, and Verma et al., 2002) and are generally associated with poor diets and inadequate intake of essential nutrients, and are correlated with stress fractures, particularly in females, and are believed to be a cause in the rising prevalence of osteoporosis (McBean et al., 1994, and Ohta et al., 2002). Surprisingly, given the above, health reports and the strong recommendation for increased calcium intake in women, children, and adolescents who are actively developing bone mass, calcium intake in developed countries is still consistently reported to be below the Food and Drug Administration's recommended consumption level (Nhanes, 1993; and McBean et al., 1994). Furthermore, persons engaged in weight bearing activities may require even greater calcium consumption than the recommended amount due to additional stimulus, which increases the calcium demand to the bones as a result of high exercise levels (Kannders et al., 1988).

Bone health depends primarily on the degree of mineralization, measured as Bone Mineral Density (BMD), which is highly affected by several environmental and dietary conditions including calcium supply and availability (Fairweather-Tait & Teucher, 2002). Dietary calcium intake and weight bearing exercise are the predominant factors beyond heredity that have been confirmed to positively influence BMD (Maktovic, 1992). Puberty is also known to play a critical key role in bone growth and development as is the individual intake of dietary calcium during adolescents (Saggese et al.; 2002). A positive correlation between calcium intake and enhancement of BMD has been demonstrated (P.S.P. 1996, Krupa 2000 and Steiner et al.; 2001), however, the correlation in young females, whose demands for calcium may be higher is not well understood. Because of the importance of calcium intake to individual health, a method for increasing calcium absorption as related in BDM would be of significant benefit.

### SUMMARY OF THE INVENTION

An object of the present invention comprises providing a method for improving calcium absorption and BMD.

These and other objects of the present invention will become apparent to those skilled in the art upon reference to the following specification and claims. The present invention pertains to a method for increasing calcium absorption and decreasing the bone loss in humans by administering to a human an effective amount of a calcium supplement in conjunction with a colostrum supplement, preferably bovine colostrum.

### DETAILED DESCRIPTION OF THE INVENTION

The purpose of the experiments that resulted in the present invention was to investigate the effect on calcium absorption as measured by BDM of calcium supplementation in combination with bovine colostrum.

Experiment 1:

In this example 60 female university students from the Home Economics Faculty of Minufiya University were selected for a double blind controlled study to determine if bovine colostrum supplementation (10 g/day) consumed over a one year period would enhance calcium absorption as measured by BMD in young (18-22 year old) female students. Subjects of this study were randomly separated into two groups and all subjects discontinued vitamin supplement intake during the study course. The first group (referred to as the colostrum group) was comprised of 35 females that received daily supplements of calcium (500 mg/day) and bovine colostrum (10 g/day). The bovine colostrum was supplied by Immuno-Dynamic, Inc., located in Perry, Iowa. The second group (referred to as the placebo group) was comprised of 25 females that received only the calcium supplementation (500 mg/day) without any bovine colostrum.

To increase compliance, subjects received refills on a monthly basis and were asked to return unused portions of the supplement. Subjects also completed a medical history questionnaire that screened for use of oral contraceptives, steroids, and anti-convulsants prior to their participation in this study. Twenty percent of the total subjects reported using oral contraceptives during the year. A baseline serum chemistry analysis was conducted on the subjects. The analysis included quantitative values for electrolytes, serum calcium, creatinine, glucose, total protein, albumin, alkaline phosphatase, and total bilirubin. The analysis failed to reject any females from the study because of a possible metabolic bone disorder.

BMD is an intensive marker of calcium bioavailability; therefore, it is used as a measure of the bone mineral status. BMD was measured at 0 (baseline), 1, 4, 8, 12, and 16 weeks on all subjects. Six skeletal sites, (lumbar spine [L1-L5], total hip, femoral neck, trochanter, ward's Triangle, and the radius) were measured utilizing the Hologic QDR 1000 watt dual x-ray absorptiometry full body scanner. Data obtained specific to the scanning instrument indicated the in vivo measurement precision for a given region of interest is better than 0.01 g/cm² and has only minimal radiation exposure (2-5 mRem).

Body composition, specifically the percentage of body fat was measured with hydrostatic weighing at three time intervals, baseline, 1 month, and 4 months. Brozek's formula was used to convert Body Density (BD) to the percentage of body fat using the data collected from hydrostatic weighing (Brozek et al.; 1963) (Brozek's formula: % BF=(4.57/BD)−4.142). Residual lung volume was also measured and was estimated as a percentage of vital capacity.

Because exercise is also a factor known to influence BMD, the VO₂ max of each subject was measured to provide descriptive data on the fitness level of each subject. VO₂ max is defined as the maximum volume of oxygen consumed by the body each minute during exercise, while breathing air at sea level. Because oxygen consumption is linearly related to energy expenditure, measuring oxygen consumption indirectly measures an individual's maximal capacity to do work aerobically. Measurements of VO₂ max were taken at baseline by placing the subjects on a treadmill and using incremental protocol to determine volitional fatigue. Each warm up and testing stage lasted for two minutes. The stages were graded, beginning at 0% and increased 2% per stage. The treadmill speed was self-selected by each subject and left constant throughout the testing stage. Both the heart rhythm and heart rate were monitored electrically using a 6 lead electrocardiogram (EKG), and the blood pressure of the subjects was monitored following each stage change. The testing was terminated upon the subject's inability to continue or when the VO₂ max failed to increase with an increase in workload.

The subjects of both groups were required to report three day dietary records and seven day activity records quarterly throughout the year duration of this study. Standardized forms were given to the subjects to be used for the dietary and activity records and all the subjects received detailed instructions regarding completion of the records. The three “typical” food consumption days that were recorded were analyzed using Nutritions III software. The software program includes 5000 foods and analyzes each food for 58 food components. Complete dietary data was available only for the first six months of the study. The analysis performed for the average calcium intake is believed to accurately reflect the calcium intake for each subject over the course of the study. Energy expenditure was also estimated from the subject's seven day activity records that were obtained quarterly. The Harris Benedict equation was used to calculate each subject's Basal Metabolic Rate (BMR). The Harris Benedict equation is as follows: 655+(9.6×weight in kilos)+(1.8×height in centimeters)−(4.7×age in years)=BMR. Workout intensity was also estimated from the reported workout records and energy expenditure was extrapolated.

The relationship between primary and secondary efficacy variables was investigated using Pearson Product-Moment Correlation and Spearman Rank Correlation and the impact of bovine colostrum supplementation on BMD was investigated using Analysis of Covariance (ANCOVA), with the average bovine colostrum intake serving as the co-variant. The Pearson Product-Moment Correlation refers to a technique for determining a correlation coefficient using continuous data such as height and weight. The Spearman Rank Correlation is a statistical technique for determining a correlation coefficient using data in the form of ranks rather than the data itself, and is often called the rank-order correlation technique. Therefore, using the above methods, the resulting statistical analysis consisted of a 2×3 ANOVA (groups×time) for BMD. A significance level of P≦0.05 (alpha=0.05) was used for these analyses.

Results:

Each of the original 60 subjects, 35 taking bovine colostrum in conjunction with the calcium supplement and 25 subjects taking a placebo with the calcium supplement completed the study. Table 1 shows the baseline age, anthropometric measures, VO₂ max, and BMD measurements for both the colostrum supplementation (CS) group and placebo group. The differences shown in Table 1 are not considered to be clinically relevant. There was no significant difference between the two groups height (P=0.11), weight (P=0.10) body mass index (BMI, P=0.46), percent body fat (P=0.89) or VO₂ max (P=0.90). The mean baseline BMD measurements of the bovine colostrum supplementation and placebo groups were not significantly different at any of the six sites evaluated. It was discovered that height and weight were significantly correlated with BMD at the lumbar spine site (r≧0.50@p<0.05) but neither the percentage of body fat nor the VO₂ max were significantly correlated with BMD.

The mean of the calculated compliance for those taking the supplement over the duration of four months, was 64±4.8% and 73±6.3%, for bovine colostrum supplementation and placebo groups respectively. Compliance data also suggests that the intake of calcium for the placebo group failed to reach the daily recommended level of calcium intake. The differential range of increase in calcium intake between colostrum and placebo groups over the course of the study was 420-644 mg, showing that subjects in the colostrum supplemented group reported a higher daily intake of calcium when compared to the placebo group. The food analysis software showed that the mean vitamin D consumption reported by the calcium plus colostrum supplementation group was greater than for the placebo group (2.7±0.4 μg) however, the difference in the mean reported consumption of vitamin D was not significant.

TABLE 1
Baseline age, anthropomorphic measures,
VO₂ max, and BMD measurements for
bovine colostrum supplementation and placebo groups.

| | Colostrum group | Placebo group | |
|---|---|---|---|
| | Mean ± SE | Mean ± SE | |
| Measurement | (N = 35) | (N = 25) | P Value |
| Age (year) | 19.0 ± 0.2 | 19.2 ± 0.3 | P = 0.05 |
| Height (cm) | 199.4 ± 1.8 | 162.3 ± 4.6 | P = 5.05 |
| Weight (kg) | 62.0 ± 0.7 | 58.1 ± 0.08 | P = 0.10 |
| Body Mass Index (BMI) | 22.0 ± 0.4 | 21.9 ± 0.5 | P = 0.46 |
| Percent Body Fat (% BF) | 24.7 ± 1.4 | 25.2 ± 1.0 | P = 0.86 |
| VO₂Max (ml/kg/min) | 51.2 ± 2.3 | 51.3 ± 1.8 | P = 0.90 |

Mean increases were noted in the BMD between treatment groups observed over the 4 month study period as shown in Table 2. In addition, other significant changes in BMD over time were shown between the two treatment groups. For example, a higher dietary bovine colostrum intake was associated with a smaller bone loss in the lumber spine (metabolically active trabecular bone). The BMD loss in the bovine colostrum plus calcium supplementation group showed a significance of (r>0.525@p<0.05). Furthermore, using the Pearson Correlation Coefficients, colostrum intake at Ward's Triangle showed a significant BMD difference (r>0.520@p<0.05), which indicated that athletes who consumed more colostrum experienced less bone loss at the Ward's triangle BMD over the year of this study. ANOVA analysis, factoring for calcium intake, corroborates this positive relationship between colostrum supplementation, calcium intake, and BMD. Also Radial BMD was observed to remain stable in the bovine colostrum supplementation group, but declined in the placebo group at 6 months and continued to remain low in the placebo group for the duration of the study.

TABLE 2
Bone mineral density measurements for individual
Hip sites for colostrum and placebo groups.

| | | | |
|---|---|---|---|
| Bone Site | Baseline | 1 Month | 4 Months |

Bone Mineral Density (gm/cm²) in Colostrum Group

| | | | |
|---|---|---|---|
| Femoral Neck | 0.866 ± 0.029 | 0.896 ± 0.030 | 0.936 ± 0.032 |
| Trochanter | 0.730 ± 0.021 | 0.795 ± 0.023 | 0.855 ± 0.023 |
| Ward's Triangle | 0.811 ± 0.033 | 0.851 ± 0.034 | 0.889 ± 0.035 |

Bone Mineral Density (gm/cm2) in Placebo Group

| | | | |
|---|---|---|---|
| Femoral Neck | 0.878 ± 0.028 | 0.847 ± 0.029 | 0.883 ± 0.030 |
| Trochanter | 0.741 ± 0.024 | 0.731 ± 0.030 | 0.760 ± 0.082 |
| Ward's Triangle | 0.805 ± 0.036 | 0.823 ± 0.031 | 0.840 ± 0.028 |

Values are in Mean ± SEM

In summary, the mean percentage of body fat and the mean BMD measurements of the six selected sites were statistically analyzed using the methods stated above. Significant differences were found between the two groups over the duration of the study with regard to BMD (see Table 3). There were significant differences in BMD between the baseline analysis and the one month analysis. There were also further significant differences noted at 4 months between the treatment groups with respect to the percentage of body fat. The Hologic QDR 1000W used to determine BMD in this study, reports a 0.5-1.0% margin of error with regard to the accuracy of its measurement. The mean BMD change and percentage of change from the baseline level for the six sites reported in this study generally fell within this margin of error. Therefore, it is assumed that the change in BMD observed between the two groups over the course of the study was not influenced by measurement error.

Table (3): Bone mineral density gm/cm² measurement for lumbar
spine, total hip, and radius of colostrum and placebo group.

| Duration | Bone Mineral Density (g/cm²) | | | | | |
|---|---|---|---|---|---|---|
| of | Colostrum Group | | | Placebo Group | | |
| Colostrum | Lumbar | | | Lumbar | | |
| Ingestion | Spine | Total Hip | Radius | Spine | Total Hip | Radius |
| Base Line | 1.01 ± 0.03 | 1.11 ± 0.08 | 0.62 ± 0.03 | 0.97 ± 0.10 | 1.03 ± 0.10 | 0.62 ± 0.08 |
| 1 Week | 1.06 ± 0.05 | 1.18 ± 0.05 | 0.65 ± 0.04 | 0.97 ± 0.21 | 1.03 ± 0.20 | 0.63 ± 0.07 |
| 4 Weeks | 1.09 ± 0.02 | 1.21 ± 0.03 | 0.69 ± 0.04 | 0.99 ± 0.17 | 1.12 ± 0.10 | 0.60 ± 0.22 |
| 8 Weeks | 1.30 ± 0.06 | 1.32 ± 0.02 | 0.74 ± 0.01 | 1.22 ± 0.25 | 1.14 ± 0.20 | 0.57 ± 0.17 |
| 12 Weeks | 1.42 ± 0.02 | 1.50 ± 0.01 | 0.80 ± 0.02 | 1.34 ± 0.30 | 1.20 ± 0.30 | 0.58 ± 0.19 |
| 16 Weeks | 1.53 ± 0.01 | 1.62 ± 0.04 | 0.94 ± 0.01 | 1.41 ± 0.10 | 1.31 ± 0.20 | 0.62 ± 0.20 |

Although BMD is an intensive marker of calcium bioavailability, its use in dietary intervention studies is restricted to periods of significant bone growth or loss. Significant improvement in BMD was achieved with the consumption of a bovine colostrum supplement in addition to the calcium supplement; therefore, the results suggest a positive correlation between increased BMD and higher calcium absorption when calcium supplementation is combined with bovine colostrum supplementation.

The participants in the study that reported a higher calcium intake with the bovine colostrum supplement demonstrated a positive BMD response at the end of the study year. Not wishing to be bound by any particular theory of operation, several factors are believed to have influenced the results. First, it should be noted that the significant difference in calcium absorption demonstrated between the measured groups is believed to have been muted due to poor compliance and due to greater reported dietary calcium consumption. Nonetheless, the significant calcium absorption observed is believed to be related to the high level of growth factors, including Insulin-like Growth Factors (IGFs) that are naturally present and abundant in bovine colostrum. Because the IGFs have pronounced stimulating roles in calcium absorption and retention, as well as muscle mass modeling in addition to body bone formation, it is proposed that the consumption of bovine colostrum along with a calcium supplement could offer long-term protection against such bone disorders as osteoporotic fractures, which occur during advanced age. In addition, IFGs include IGF-binding proteins and other hormonally active peptides, which are believed to be important in stimulating the optimal growth and development in human serum.

It is further postulated that due to the modulatory effects of bovine colostrum on cytokines (Interleukins 1 & 6) and the production and release of tumor-necrosis factors-α (TNF-α) in humans, bovine colostrum will offer protection against bone loss through improved calcium absorption, perhaps through modulation of intestinal epithelial cell deterioration. Furthermore, bovine colostrum is reported to have a high level of the most important dietary bioactive oligosaccharides and glyco-conjugates that are believed to have pronounced cooperative effects in the prevention of bone osteopenia. Therefore, the presence of bovine colostrum presumably increases the availability of metabolites involved in the control of bone mineral loss, and involved in the regulation of calcium absorption and loss.

It has previously been demonstrated that bone density accrual appears to plateau in humans by the age of 20; therefore, BMD accrual is seen most dramatically in the 12-15 year age group. Also of concern when analyzing the results is the fact that during the selected age of the females in the experiment, young women in this stage of life frequently participate in fad diets that are often deficient in essential nutrients such as calcium. Young females are also vulnerable to eating disorders that may precipitate menstrual irregularities, thus further negatively impacting the bone mineral accrual at a crucial point in its development. Therefore, it is postulated that supplementation of calcium in combination with bovine colostrum may be more productive in younger populations, or populations with better dietary habits. Furthermore, it may be that the present invention may be more useful in preventing bone loss than in increasing BMD in older populations, including post-menopausal women.

The absorption of dietary calcium shows a gradual decline in adulthood, therefore the incorporation of an adequate amount of bovine colostrum in conjunction with the calcium supplementation to enhance and improve the bone mineral content in childhood will help maintain the bone mineral content in adults. This confirms the necessity of bovine colostrum supplementation throughout the life stages that would benefit from a high calcium intake, such as childhood, puberty, pregnancy, and pre-school life stages. This is to ensure the ideal and optimal bone growth and development in addition to offering protection against the risk of osteoporotic fractures during advanced age. In addition, the maintenance of an adequate calcium level throughout life could minimize the risks of bone-loss disorders. Maximum BMD benefit from enhanced dietary calcium intake or calcium supplementation may best be achieved by bovine colostrum ingestion focusing on other age groups, including pre-school children, to insure adequate and proper calcium intake and therefore ideal and maximum bone growth and development. There has long been evidence supporting the positive association between calcium intake and bone mass accrual in females as well as evidence supporting an increase in the recommended dietary allowance (RDA) for calcium in children, adolescents, and young adults. Calcium intake should be monitored along with other dietary factors affecting the balance between absorption and excretion.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

Claiming:

1. A method for increasing calcium absorption in humans comprising administering to a human an effective amount of a calcium supplement in conjunction with a colostrum supplement until calcium absorption is increased.

2. The method for increasing calcium absorption as defined in claim 1, wherein the colostrum consumed is bovine colostrum.

3. The method for increasing calcium absorption as defined in claim 2, wherein the amount of bovine colostrum administered is between 1 gram and 15 grams daily.

4. The method for increasing calcium absorption as defined in claim 1, wherein the amount of calcium supplement administered is between 200 grams and 800 grams daily.

5. The method for increasing calcium absorption as defined in claim 1, wherein the humans are below the age of 20.

6. The method of increasing the calcium absorption as defined in claim 5 wherein the humans are females.

7. A method for decreasing bone loss in humans comprising administering to a human an effective amount of a calcium supplement in conjunction with a colostrum supplement for decreasing bone loss.

8. The method for decreasing bone loss as defined in claim 7, wherein the colostrum consumed is bovine colostrum.

9. The method for decreasing bone loss as defined in claim 8, wherein the amount of bovine colostrum administered is between 1 gram and 15 grams daily.

10. The method for decreasing bone loss as defined in claim 7, wherein the amount of calcium supplement administered is between 200 grams and 800 grams daily.

11. A method for increasing calcium absorption and decreasing bone loss in the elderly, comprising administering to an elderly person an effective amount of a calcium supplement in conjunction with a colostrum supplement for increasing calcium absorption and decreasing bone loss.

12. The method for increasing calcium absorption and decreasing bone loss as defined in claim 11, wherein the colostrum consumed is bovine colostrum.

13. The method for increasing calcium absorption and decreasing bone loss as defined in claim 12, wherein the amount of bovine colostrum administered is between 1 gram and 15 grams daily.

14. The method for increasing calcium absorption and decreasing bone loss as defined in claim 11, wherein the amount of calcium supplement administered is between 200 grams and 800 grams daily.

15. A method for increasing calcium absorption and decreasing bone loss in post-menopausal women comprising, administering to a post-menopausal woman an effective amount of a calcium supplement in conjunction with a colostrum supplement for increasing calcium absorption and decreasing bone loss.

16. The method for increasing calcium absorption and decreasing bone loss as defined in claim 15, wherein the colostrum consumed is bovine colostrum.

17. The method for increasing calcium absorption and decreasing bone loss as defined in claim 16, wherein the amount of bovine colostrum administered is between 1 gram and 15 grams daily.

18. The method for increasing calcium absorption and decreasing bone loss as defined in claim 15, wherein the amount of calcium supplement administered is between 200 grams and 800 grams daily.

19. A supplement for increasing calcium absorption and decreasing bone loss comprising an effective amount of calcium in conjunction with colostrum, the supplement administered to humans in order to increase calcium absorption and decrease bone loss.

20. The supplement for increasing calcium absorption and decreasing bone loss as defined in claim 19, wherein the colostrum in the supplement is bovine colostrum.

21. The supplement for increasing calcium absorption defined in claim 18, wherein the amount of bovine colostrum in the supplement is between 1 gram and 15 grams daily.

22. The supplement for increasing calcium absorption as defined in claim 19, wherein the amount of calcium in the supplement is between 200 grams and 800 grams daily.

23. A method of reducing the risk of osteoporosis in humans, comprising administering to a human an effective amount of a calcium supplement in conjunction with a colostrum supplement until the risk of osteoporosis is reduced.

24. A method of reducing the risk of bone-loss disorders in humans, comprising administering to a human an effective amount of a calcium supplement in conjunction with a colostrum supplement until the risk of bone-loss disorders is reduced.
